(19) United States
(12) Patent Application Publication
(10) Pub. No.: US 2026/0026882 A1
(43) Pub. Date: Jan. 29, 2026

(54) SYSTEM AND METHOD OF USING A HEALTHY SIDE OF A BRAIN AS A TRACTOGRAPHY QUANTIFICATION STANDARD

(71) Applicant: Synaptive Medical Inc., Toronto (CA)

(72) Inventors: Alicia McNeely, Toronto (CA); Wesley Bryan Hodges, London (CA); Sebastian Koga, Covington, LA (US); Tim Hayes, Toronto (CA)

(21) Appl. No.: 19/284,494

(22) Filed: Jul. 29, 2025

Related U.S. Application Data
(60) Provisional application No. 63/676,478, filed on Jul. 29, 2024.

(51) Int. Cl. A61B 34/10; A61B 5/00; A61B 5/055; G06T 7/00; G06T 7/11; G06T 7/68; G16H 20/40; G16H 70/60

(52) U.S. Cl. CPC A61B 34/10; A61B 5/0042; A61B 5/055; A61B 5/742; G06T 7/0016; G06T 7/11; G06T 7/68; G16H 20/40; G16H 70/60; G06T 2207/10088; G06T 2207/30016

(57) ABSTRACT

A system and method is provided using a healthy side of a brain as tractography quantification standard in surgical planning. Tools are used to compare tractography in the pathological (ipsilateral) hemisphere to the contralateral hemisphere. The tool identifies bundles in the ipsilateral and contralateral hemisphere using surgical planning software that includes an auto-segmentation feature, and provides a ratio of their symmetry, and metric comparison. This gives users a more objective and clinically relevant way of interpreting the results of diffusion quantification. It also provides a metric that can be compared longitudinally as a hemisphere recovers after tumor resection, and brain symmetry is restored.

The instant application claims priority as a non-provisional to U.S. application Ser. No. 63/676,478 filed on Jul. 29, 2024, presently pending, the contents of which are hereby incorporated by reference.

### TECHNICAL FIELD

The field of the invention is systems and methods for surgical planning, in particular, systems related to neurosurgery.

### BACKGROUND

Tractography is currently used in surgical planning and navigation as a visualization tool. Users are able to qualitatively assess the tracts to determine the best surgical approach. Now that tractography is becoming easier to access because of automation and the education and clinical evidence of its value is growing, there is a potential for it to be used more broadly, and allow for expansion of use before surgery (for example as a diagnostic, used by radiology), after surgery (prognostic, used to track patient recovery), and in other disciplines such as neurology, for diagnostics and prognostics, or radiation dose planning.

In order for this expansion to happen, users need the ability to measure characteristics of tracts, such as the amount of diffusion in a given nerve bundle, in order to quantify the “health” of the bundle, and investigate this over time. Adding the ability to quantify tracts rather than just visualize them, will allow for expansion of the application of tractography to markets outside of pre-and intra-operative use.

Once available, users will need a control or standardized way of determining whether a bundle is healthy or damaged based on the quantifiable value presented by the software. This is not something readily available because tractography quantification is a novel tool for clinical use, and the methodology of generating tracts (including underlying imaging and algorithm used in post-processing) are not standardized. Users need a standardized and clinically viable way of determining how the metrics compare to normal, so they can use them to make decisions.

### BRIEF DESCRIPTION OF THE DRAWINGS

FIG. 1 is a diagram illustrating a pre-operative deviation of pathological hemisphere of the brain.

FIG. 2 is a diagram illustrating a post-operative image of the brain.

FIG. 3 is a diagram illustrating a graphical user interface of quantitative diffusion metrics.

### SUMMARY

A system and method is provided for using a healthy side of a brain as tractography quantification standard in surgical planning. Tools are used to compare tractography in the pathological (ipsilateral) hemisphere to the contralateral hemisphere. The tool will identify bundles in the ipsilateral and contralateral hemisphere using surgical planning software that includes an auto-segmentation feature, and provide a ratio of their symmetry, and metric comparison. This gives users a more objective and clinically relevant way of interpreting the results of diffusion quantification. It will also provide a metric that can be compared longitudinally as anatomy recovers after tumor resection, and brain symmetry is restored.

In some aspects, the techniques described herein relate to a method for quantifying a pathology status of an ipsilateral side of a nervous system, including: obtaining an ipsilateral tractography data of the ipsilateral side of the nervous system; converting the ipsilateral tractography data to an ipsilateral quantitative data; obtaining a contralateral tractography data of a contralateral side of the nervous system; converting the contralateral tractography data to a contralateral quantitative data; comparing the ipsilateral quantitative data to the contralateral quantitative data; determining if there is one or more difference between the ipsilateral quantitative data and the contralateral quantitative data; and calculating the pathology status of the ipsilateral side of the nervous system using the difference.

In some aspects, the techniques described herein relate to a system for quantifying a pathology status of an ipsilateral side of a nervous system including: a magnetic resonance imaging (MRI) device for obtaining an ipsilateral tractography data of the nervous system and a contralateral tractography data of the nervous system; a computer storage system for storing the ipsilateral tractography data and the contralateral tractography data; a computer processor for converting the ipsilateral tractography data to an ipsilateral quantitative data and the contralateral tractography data to a contralateral quantitative data; comparing the ipsilateral quantitative data to the contralateral quantitative data; determining if there is one or more difference between the ipsilateral quantitative data and the contralateral data; calculating the pathology status of the ipsilateral side of the nervous system using the difference; a computer memory for storing the difference and the pathology status; and a computer display for providing the pathology status to a user.

### DETAILED DESCRIPTION

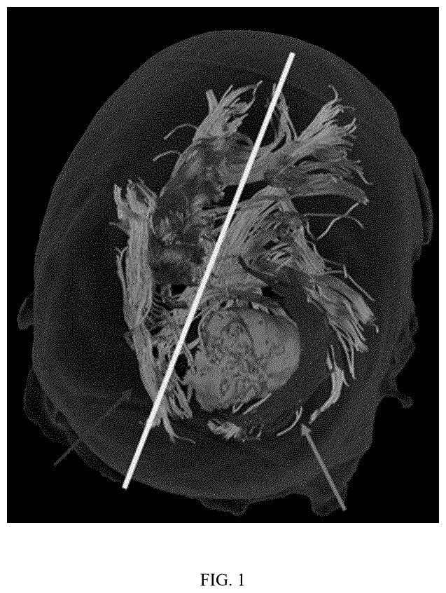

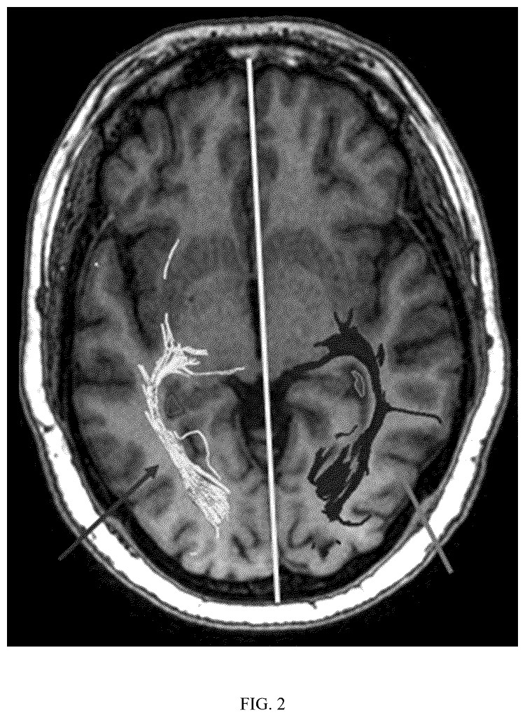

This disclosure provides users with a tool to compare tractography in the pathological (ipsilateral) hemisphere to the contralateral hemisphere, in order to use the contralateral hemisphere as a control for the ipsilateral hemisphere, as illustrated in FIGS. 1 and 2.

As tractography becomes quantifiable by a tractography software, users need a control or standardized method of determining whether a bundle is healthy or damaged based on the quantifiable value presented by the software. This is not something readily available because tractography quantification is a novel tool for clinical use, and the methodology of generating tracts (including underlying imaging and algorithm used in post-processing) are not standardized. Users need a standardized and clinically viable way of determining how the metrics of a pathological tissue compare to normal, so they can use the metrics to make medical decisions and analyze outcomes of medical procedures.

FIG. 1 is a diagram illustrating a pre-operative deviation of a pathological (ipsilateral) hemisphere of the brain. FIG. 1 shows deviation of the pathological hemisphere from the midline due to tumor displacement of tracts. The arrows point to the bundle of interest, the optic radiations.

FIG. 2 is a diagram illustrating a post-operative image of the brain. According to FIG. 2, deviation of the pathological (ipsilateral) hemisphere is resolved and symmetry is regained. The ability to quantify the diffusion in the bundles of the ipsilateral hemisphere pre-and post-operatively, and compare quantitative tractography data of the ipsilateral and contralateral hemispheres would provide an internal control, as well as prognostic information after surgery.

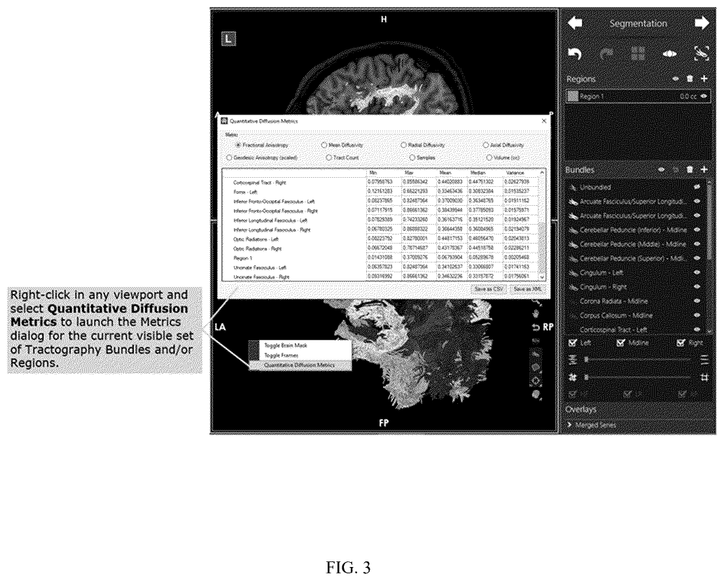

FIG. 3 is a diagram illustrating a graphical user interface of quantitative diffusion metrics.

### Quantitative Diffusion Metrics Applications

Tractography is becoming easier to access because of automation, and the education and clinical evidence of its value is growing. There is a potential for it to be used more broadly and allow for expansion of use before surgery (diagnostic, used by radiology), after surgery (prognostic, used to track patient recovery), and in other disciplines such as neurology, for diagnostics and prognostics, or radiation dose planning.

In order for this expansion to happen, users need the ability to measure characteristics of tracts, such as the amount of diffusion in a given bundle, in order to quantify the “health” of the bundle and investigate this over time. Adding the ability to quantify tracts rather than just visualize them provides a successful expansion of the application of tractography to markets outside of pre-and intra-operative use.

According to this disclosure, a research version of a tractography software (Modus Plan™) allows users to quantify data including, as a non-limiting example, diffusion and count in the nerve bundles to create a more objective measure of the nerve bundle health and robustness. The metrics included are:
Fractional Anisotropy
Mean diffusivity
Radial diffusivity
Axial diffusivity
Geodesic anisotropy.
Tract Count

Additional metrics can be included in the quantification metrics, based on feedback from clinicians. Metrics that are useful and sensitive enough to detect function and health of the bundles and explore the link to patient outcomes and recovery are prioritized.

The tool identifies bundles in the ipsilateral and contralateral hemisphere using surgical planning software that includes an auto-segmentation feature, and provides a ratio of the symmetry of the bundles between each hemisphere, and metric comparison.

This gives users a more objective and clinically relevant way of interpreting the results of diffusion quantification. It also provides a metric that can be compared longitudinally as anatomy recovers after tumor resection for example, and brain symmetry is restored.

Tractography is not currently used in a qualitative way, and there are no clinical tools known that use the contralateral hemisphere as a control. This new system and method expands the application of tractography.

A method for quantifying a pathology status of an ipsilateral side of a nervous system includes obtaining an ipsilateral tractography data of the ipsilateral side of the nervous system using a magnetic resonance imaging device (MRI) and converting the ipsilateral tractography data to ipsilateral quantitative data. The contralateral tractography data of a contralateral side of the nervous system is likewise obtained by MRI and converted to contralateral quantitative data. The ipsilateral quantitative data is compared to the contralateral quantitative data to determine if there is one or more difference between the ipsilateral quantitative data and the contralateral quantitative data. If there is a difference or multiple differences between the ipsilateral quantitative data and the contralateral quantitative data, the differences are used to calculate the pathology status of the ipsilateral side of the nervous system using the difference.

One example of a method to convert the ipsilateral and contralateral tractography data to the ipsilateral and contralateral quantitative data is to identify at least one ipsilateral bundle of the nervous system and at least one corresponding contralateral bundle of the nervous system for comparison of the bundles. The bundles may be identified using an auto-segmentation feature of the tool, for example a software program. The ipsilateral and contralateral bundles of the nervous system may be connecting nerve fibers.

The ipsilateral quantitative data may be compared to the contralateral quantitative data by providing a ratio of a symmetry between the one or more ipsilateral bundle and the one or more contralateral bundle.

The pathology status of the ipsilateral side of the nervous system may be calculated longitudinally, for example at time points after surgery, as the ipsilateral side of the nervous system is restored to have a symmetry with the contralateral side.

A system is also provided for quantifying a pathology status of an ipsilateral side of a nervous system. The system includes an MRI device for obtaining an ipsilateral tractography data and a contralateral tractography data of the nervous system. A computer storage system may be used for storing the ipsilateral tractography data and the contralateral tractography data. A computer processor converts the ipsilateral tractography data to an ipsilateral quantitative data and the contralateral tractography data to a contralateral quantitative data. The ipsilateral quantitative data is compared to the contralateral quantitative data to determine if there is one or more difference between the ipsilateral quantitative data and the contralateral data. The pathology status of the ipsilateral side of the nervous system can be calculated using the difference between the quantitative data of each side. The difference and calculated pathology status can be stored in a computer memory and a computer display provides the pathology status to a user.

The conversion of the ipsilateral and contralateral tractography data to the ipsilateral and contralateral quantitative data may be done by the computer program using an auto-segmentation process to identify at least one ipsilateral bundle of the nervous system and a corresponding at least one contralateral bundle of the nervous system. The computer processor can compare the ipsilateral quantitative data to the contralateral quantitative data and provide a ratio of a symmetry between the one or more ipsilateral bundle and the one or more contralateral bundle. The bundles of the nervous system may be connecting nerve fibers.

The system may also be used to determine the pathology status of the ipsilateral side over a period of time, i. e. longitudinally, as the ipsilateral side of the nervous system is restored to a symmetry with the contralateral side, for example after a surgical procedure.

Implementations disclosed herein provide systems, methods and apparatus for generating or augmenting training data sets for machine learning training. The functions described herein may be stored as one or more instructions on a processor-readable or computer-readable medium. The term “computer-readable medium” refers to any available medium that can be accessed by a computer or processor. By way of example, and not limitation, such a medium may comprise RAM, ROM, EEPROM, flash memory, CD-ROM or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor. A “module” can be considered as a processor executing computer-readable code.

A processor as described herein can be a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, or microcontroller, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, any of the signal processing algorithms described herein may be implemented in analog circuitry. In some embodiments, a processor can be a graphics processing unit (GPU). The parallel processing capabilities of GPUs can reduce the amount of time for training and using neural networks (and other machine learning models) compared to central processing units (CPUs). In some embodiments, a processor can be an ASIC including dedicated machine learning circuitry custom-build for one or both of model training and model inference.

The disclosed or illustrated tasks can be distributed across multiple processors or computing devices of a computer system, including computing devices that are geographically distributed. The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.” While the foregoing written description of the system enables one of ordinary skill to make and use what is considered presently to be the best mode thereof, those of ordinary skill will understand and appreciate the existence of variations, combinations, and equivalents of the specific embodiment, method, and examples herein. The system should therefore not be limited by the above-described embodiment, method, and examples, but by all embodiments and methods within the scope and spirit of the system. Thus, the present disclosure is not intended to be limited to the implementations shown herein but is to be accorded the widest scope consistent with the principles and novel features disclosed herein.

What is claimed is:

1. A method for quantifying a pathology status of an ipsilateral side of a nervous system, comprising:
obtaining an ipsilateral tractography data of the ipsilateral side of the nervous system;
converting the ipsilateral tractography data to an ipsilateral quantitative data;
obtaining a contralateral tractography data of a contralateral side of the nervous system;
converting the contralateral tractography data to a contralateral quantitative data;
comparing the ipsilateral quantitative data to the contralateral quantitative data;
determining if there is one or more difference between the ipsilateral quantitative data and the contralateral quantitative data; and
calculating the pathology status of the ipsilateral side of the nervous system using the difference.

2. The method of claim 1, wherein converting the ipsilateral and contralateral tractography data to the ipsilateral and contralateral quantitative data comprises identifying at least one ipsilateral bundle of the nervous system and an at least one corresponding contralateral bundle of the nervous system.

3. The method of claim 2, wherein the at least one bundle of the nervous system and the at least one corresponding contralateral bundle of the nervous system comprise connecting nerve fibers.

4. The method of claim 2, wherein comparing the ipsilateral quantitative data to the contralateral quantitative data comprises providing a ratio of a symmetry between the at least one ipsilateral bundle and the at least one contralateral bundle.

5. The method of claim 2, wherein identifying bundles in the ipsilateral and contralateral nervous system comprises an auto-segmentation feature of a software.

6. The method of claim 1 wherein the pathology status of the ipsilateral side of the nervous system is calculated longitudinally as the ipsilateral side of the nervous system is restored to a symmetry with the contralateral side.

7. A system for quantifying a pathology status of an ipsilateral side of a nervous system comprising:
a magnetic resonance imaging (MRI) device for obtaining an ipsilateral tractography data of the nervous system and a contralateral tractography data of the nervous system;
a computer storage system for storing the ipsilateral tractography data and the contralateral tractography data;
a computer processor for
converting the ipsilateral tractography data to an ipsilateral quantitative data and the contralateral tractography data to a contralateral quantitative data;
comparing the ipsilateral quantitative data to the contralateral quantitative data;
determining if there is one or more difference between the ipsilateral quantitative data and the contralateral data;
calculating the pathology status of the ipsilateral side of the nervous system using the difference;
a computer memory for storing the difference and the pathology status; and
a computer display for providing the pathology status to a user.

8. The system of claim 7, wherein converting the ipsilateral and contralateral tractography data to the ipsilateral and contralateral quantitative data comprises the computer program using an auto-segmentation process to identify at least one ipsilateral bundle of the nervous system and a corresponding at least one contralateral bundle of the nervous system.

9. The system of claim 8, wherein the at least one ipsilateral bundle of the nervous system and the corresponding at least one contralateral bundle of the nervous system comprise connecting nerve fibers.

10. The system of claim 7, wherein comparing the ipsilateral quantitative data to the contralateral quantitative data comprises the computer program providing a ratio of a symmetry between the at least one ipsilateral bundle and the at least one contralateral bundle.

11. The system of claim 6. wherein the pathology status of the ipsilateral side of the nervous system is calculated longitudinally as the ipsilateral side of the nervous system is restored to a symmetry with the contralateral side.
